(19) United States
(12) Patent Application Publication
(10) Pub. No.: US 2026/0248432 A1
(43) Pub. Date: Aug. 27, 2026

(54) HIGH SURFACE AREA ELECTRODES FOR INVASIVE MEDICAL DEVICES

(71) Applicant: NEURONOFF, INC., Cleveland, OH (US)

(72) Inventors: Manfred FRANKE, Cleveland, OH (US); Stephan NIEUWOUDT, Cleveland, OH (US)

(73) Assignee: NEURONOFF, INC., Cleveland, OH (US)

(21) Appl. No.: 18/827,996

(22) PCT Filed: Mar. 13, 2023
(86) PCT No.: PCT/US2023/015131

§ 371 Date: Sep. 9, 2024

Related U.S. Application Data
(60) Provisional application No. 63/319,379, filed on Mar. 13, 2022.

(51) Int. Cl. A61B 5/263; A61B 5/293; A61B 5/37; A61B 18/00; A61B 18/14; A61N 1/05

(52) U.S. Cl. CPC A61B 5/263; A61B 5/293; A61B 5/37; A61B 18/1477; A61N 1/0534; A61N 1/0553; A61N 1/056; A61B 2018/00577; A61B 2018/1437; A61B 2018/144; A61B 2562/0209

(57) ABSTRACT

Disclosed is a high surface area electrode assembly, for use as an invasive device, which is porous and has significantly more surface area over prior art invasive electrodes. Various embodiments incorporate a high surface area electrode into a nonconductive layer or length or a DBS-like versions suitable for deep brain stimulation and the like.

### PRIORITY AND INCORPORATION STATEMENT

This application claims priority to, and the full benefit of, U.S. provisional patent application No. 63/319,379 filed Mar. 13, 2022.

The invention here comprises one or more high surface area electrodes such as those disclosed in international applications PCT/US20/61374 (the “374 application”) filed Nov. 19, 2020, PCT/US21/33007 (the “'007 application”) filed May 18, 2021 and PCT/US21/33265 (the “265 application”) filed May 19, 2021, and these applications are incorporated herein in their entirety as if set forth herein. This application also adopts reference numbers from the '007 and '265 applications.

### ASPECTS OF THE INVENTION

In addition to the inventions for minimally invasive use of the high surface area electrodes (sometimes herein called “the Injectrode”) through needle injection described in the '374, '007 and '265 applications, these high surface area electrodes can also be incorporated into invasive or other medical devices, herein called the high surface area electrode assembly 91. As used herein the high surface area electrode 92 is one selected from the group consisting of a helical wire rope structure 1 (e.g., FIGS. 2-A and 2B of the '007 application), a wire rope 22 (FIG. 1 of the '007 application), a wire mesh 93, and folded, rolled, twisted, or braided non-helical wire structures (e.g., FIGS. 11(d), 12(d), 13(c), 17(d) and 18(b) of the '374 application). Herein, “high surface area electrode” 92 is used interchangeably with the group consisting of a helical wire rope structure 1, a wire rope 22, a wire mesh 93, and folded, rolled, twisted, or braided non-helical wire structures. All of the embodiments of the invention are highly porous, allowing bodily fluid to flow through and around the wire composing them.

As used herein, “invasive electrode” includes an implantable electrode which cannot be injected with a needle and requires an incision by scalpel or similar instrument.

The present invention provides a novel and significant increase in active surface area for the transfer of electrical or other energy to or from a metal electrode near a tissue target and/or in the interstitial fluid. Prior art electrode assemblies are shown in U.S. patent application Ser. No. 16/439,323, FIGS. 3A-4B , and prior art smooth surface electrodes are also shown in FIGS. 2A-2B and FIGS. 5-7 in Ser. No. 16/439,323. Smooth surfaces may be curved but they, like the planar surface electrodes, share the lower surface area of the electrode compared to the invention herein. Advantages of the invention herein include (1) lower impedance stimulation than planar/smooth electrode surfaces, (2) lower impedance ablation, (4) larger charge transfer capabilities as well as heat generation and transfer capabilities for ablation, (5) larger charge injection and charge storage capabilities within the Helmholtz double layer of the electrode for (temporary) direct current transfer or charge injection, and serves as an alternative to surface modification to increase surface area, (6) extending battery life or reducing battery size because of lower impedance of the electrodes and (7) reducing the size of cardiac pacemakers or implantable cardioverter defibrillators (ICDs) because of lower voltage needs and thereby smaller capacitors (which make up about ⅓ of the inside of an ICD) to have the power to pace or cardiovert a heart, (8) increasing resolution and sensitivity of sensing electrodes due to larger surface area, (9) reducing noise for sensing electrodes, (10) increasing capacitive vs. resistive coupling to the surrounding medium, e.g. interstitial or cerebrospinal fluid, (11) enabling large amount of charge injection without leaving the water window which increases safety of electrodes for traditional neurostimulation applications as well as enables larger charge injection for temporary use cases such as monophasic charge injection for seconds prior to charge balancing for e.g. temporary nerve block applications, and (12) increasing the sensitivity, longevity and reliability of the system if the wire of the high surface area electrode is further functionalized for sensing of biomedical, biological, pharmacological and other agents.

The portion of the lead electrochemically not exposed inside the insulation is connected to the electrochemically exposed electrode portion of the device can be manufactured in the same way, or instead can be connected, e.g., by crimping, welding or similar process to the electrochemically exposed electrode portion of the device and still be located in an electrically insulated area such as underneath a layer of silicone. This allows the entire high surface area electrode to remain mechanically very flexible and attain a similar flexibility and stretchability as the often very flexible non-conductive substrate (such as silicone sheet). This allows for a very flexible overall electrode structure.

Certain portions of the device may be separately coated in an insulating material such as polyether ether ketone (PEEK), parylene C or others to define specific regions of the device that are able to remain electrochemically inactive and thus not participating in the charge transfer process as compared to the uncoated areas of the device that are able to easily wick with bodily fluids once implanted.

These advantages of the invention also apply to the minimally invasive versions of the Injectrode as in the '374, '007 and '265 applications, but incorporation of the high surface area electrode into invasive electrode assemblies is new. The nonconductive layer or length, as distinguished from a thin coating for insulation purposes, provides great stability for placing the high surface electrode via, for example, an incision when injection by a needle is not permissible. Essentially, the present invention provides stability for the high surface area electrode wherever it is used in or with the body. The invention is also well suited for flattened electrodes such as, for example, ECOG electrodes for interfacing cortical brain structures or cuff electrodes for interfacing with cylindrical structures like peripheral nerves.

The high surface area electrode also allows portions of the porous electrode interface to be closer to the neurons of interest as some threads will separate in a small loop (between 0.00001 and 0.5 mm) from the bulk of the high surface area electrode during or post implantation due to the spring characteristics of the underlying high surface area electrode.

The device may be utilized to create electrode portions for cylindrical needles, leads or paddle leads as used in spinal cord stimulation (SCS), peripheral nerve stimulation (PNS) and deep brain stimulation (DBS) applications. It may be utilized to create electrode portions for cylindrical and non-cylindrical stimulators as used in SCS, PNS or DBS, and cranial nerve applications such as vagal nerve stimulation (VNS). It may be utilized to create electrode portions for electrocorticogram (ECoG) electrodes. It may be utilized to create electrode portions for leads and stimulators in cardiac applications. Use cases may range from sensing to stimulation to temporary block and permanent block or tissue ablation. The device may be used for short term, or acute, as well as chronic placement into a living body.

### BRIEF DESCRIPTION OF THE FIGURES

FIG. 1 is an image of a portion of a helical wire rope structure electrode.

FIG. 2 is an image of a wire mesh electrode.

FIGS. 3A-3C depict a high surface area electrode assembly in a flattened embodiment. The high surface area electrode here is a helical wire rope structure, and the detail of the coils and strands is as shown in FIG. 1.

FIGS. 4A-4B depict a high surface area electrode assembly in a flattened embodiment with an undulating pattern of embodiment of the high surface area electrode. The high surface area electrode here is a helical wire rope structure, and the detail of the coils and strands is as shown in FIG. 1.

FIG. 5A depicts the high surface area electrode, as does FIG. 5B with support wires added.

FIGS. 6A-6B depict the high surface area electrode embedded in a polymer bottom layer.

FIGS. 7A-7B depict a portion of an embodiment of a DBS-like high surface area electrode assembly, showing the seating of the high surface area electrode around a smaller diameter.

FIGS. 8A-8C depict embodiments of the high surface area electrode suitable for an ablation needle.

FIGS. 9A-9C depict an embodiment of the high surface area electrode suitable for a nerve cuff electrode.

### ADDITIONAL ASPECTS OF THE INVENTION

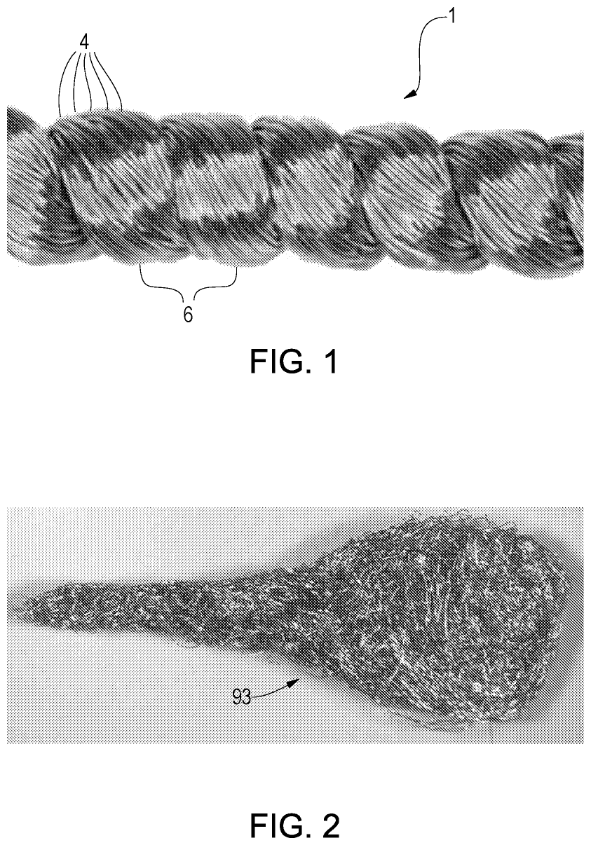

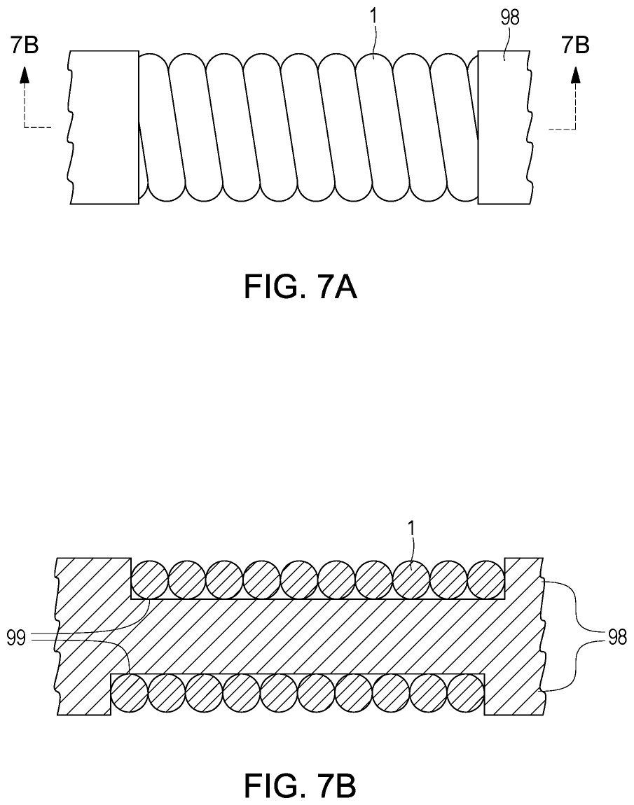

FIG. 1 is a close up of a photo of a portion of a helical wire rope structure 1 (as shown and described in the '007 and '265 applications) which is only one embodiment of the high surface area electrode 92. When uncoated with a nonconductive layer, the many strands 4 making up the coils 6 are porous in that they are exposed directly to the tissue or interstitial fluid on the exterior and the interior because of the porosity and, in some embodiments, the hollow core 5 (as shown in FIGS. 7-A, 8, 9-A, 9-B and 10 of the '007 application). Additionally, fluid flows even into coated portions from the uncoated ends or through openings in the intermittent coating. Note the irregular shape of the coils and pattern of the wire strands. Neither the coils nor the strands are bound or glued together, so porosity also results from the bodily fluid filling the many openings and pathways between them which are further expanded by movement of the body. All of this porous surface area exposed to the body's own fluid greatly expands the Helmholtz double layer over prior art invasive electrodes.

FIG. 2 is a high surface area electrode as a wire mesh 93 (as made from a process shown in part in FIGS. 19A-19D of the '374 application) and formed of conductive wire. This mesh is also highly porous. This electrode can be incorporated into an electrode assembly as described herein.

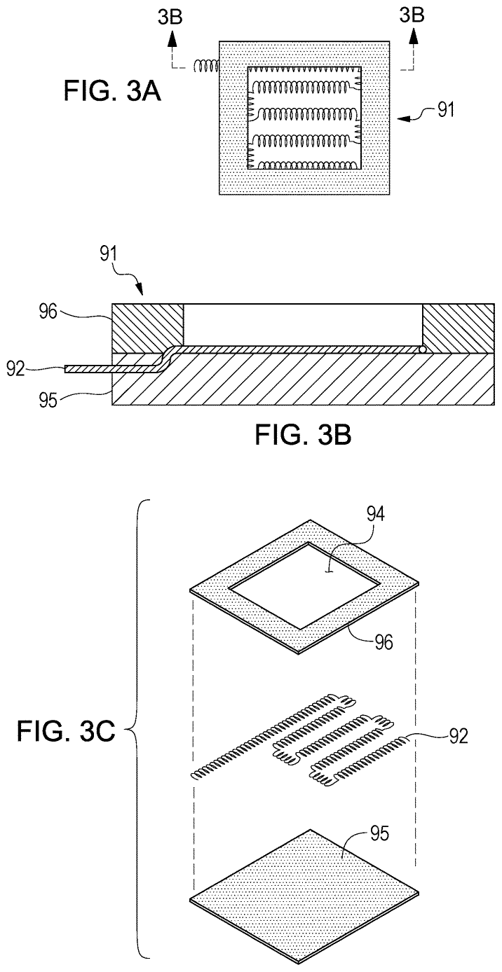

Aspects of a planar embodiment of the high surface area electrode assembly 91 are depicted in FIGS. 3A-3E. 3A is a plan view showing a high surface area electrode 92 in an open window 94. Here, as elsewhere in the figures, the high surface area electrode is shown only in its general shape but does not include the detail and texture of this component which is shown, for example in the image of FIG. 1 herein. FIG. 3B is a cross-section showing the bottom layer 95, the upper layer 96, and the path of the high surface area electrode 92 (again, without the detail in FIG. 1). In an exploded view, 3C shows the upper layer 96 surrounding an open window 94, the high surface area electrode 1, and the bottom layer 95.

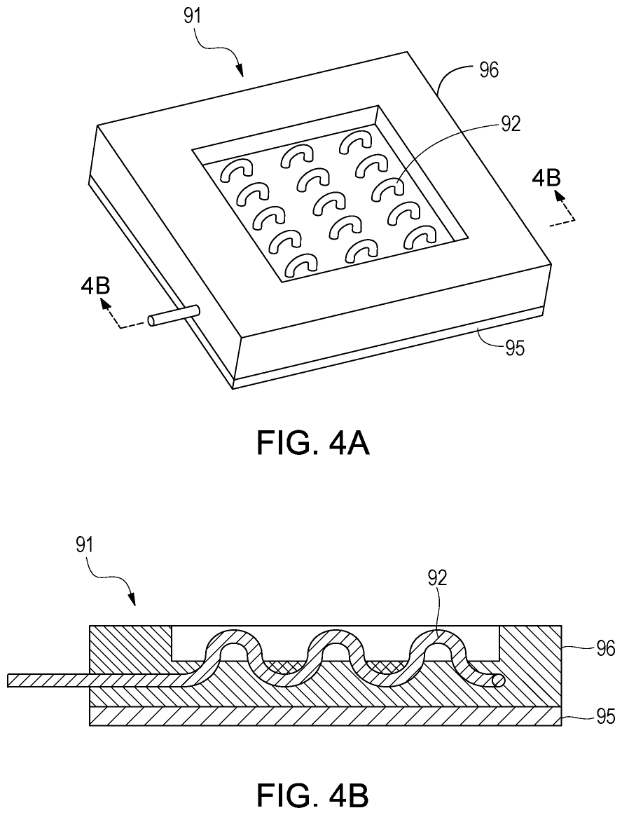

FIG. 4A is a perspective drawing of a flattened embodiment of the high surface area electrode assembly 91 with the high surface area electrode 92 partially embedded in the bottom layer 95, that is, undulating in and out of the bottom layer. FIG. 4B is a section view of the embodiment at line 4B-4B in FIG. 4A.

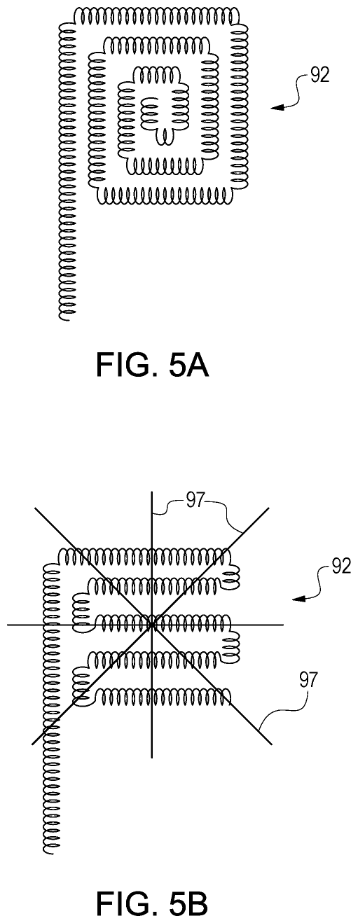

As shown in FIGS. 5A-5B, a planar embodiment of the high surface area electrode 92 to be placed or embedded between the bottom and upper layers of nonconductive material. FIG. 5A depicts a spiral high surface area electrode 92 and FIG. 5B a somewhat random or meandering pattern. Likewise, FIGS. 4A and 4B may also comprise wire rope as the high surface area electrode in these or any number of other patterns. In FIG. 4B, an additional set of support wires 97 overlay to add strength to the high surface area electrode 92.

The high surface area electrode replaces the disc or foil (smooth or relatively so compared to the invention) that normally provides the electrode interface in prior art electrodes. The prior art disc or foil is sandwiched between two non-conductive planes made from silicone, or a plastic such as polyimide or others. Because the high surface area electrode has a depth to itself compared to the foil, in some embodiments there is sufficient depth of space within the planes by which the high surface area electrode is held in place. One method of constructing the invention is filling the open window in the upper (tissue target facing) layer. Another method is filling an intermediate layer between the upper and lower layers with a high surface area electrode. Another method is filling an indentation in the bottom layer with a high surface area electrode. Another method is using single strand meshes in the same way as the foil would be used or overlaying several meshes to form the charge injecting porous volume. In FIGS. 5A and 5B various forms of anchoring are shown. In 5B a high surface area electrode is anchored by support wires 97 in turn encased in the planes, while in 5B the high surface area electrode has sufficient structural integrity to be partially encased or submerged in silicone prior to curing. Other embodiments similar to 5A and 5B employ a braid or other wire structure described in the '374 application. Or, the wire mesh 93 as in FIG. 2 fills the open window, and portions of it are embedded or sandwiched between the two layers.

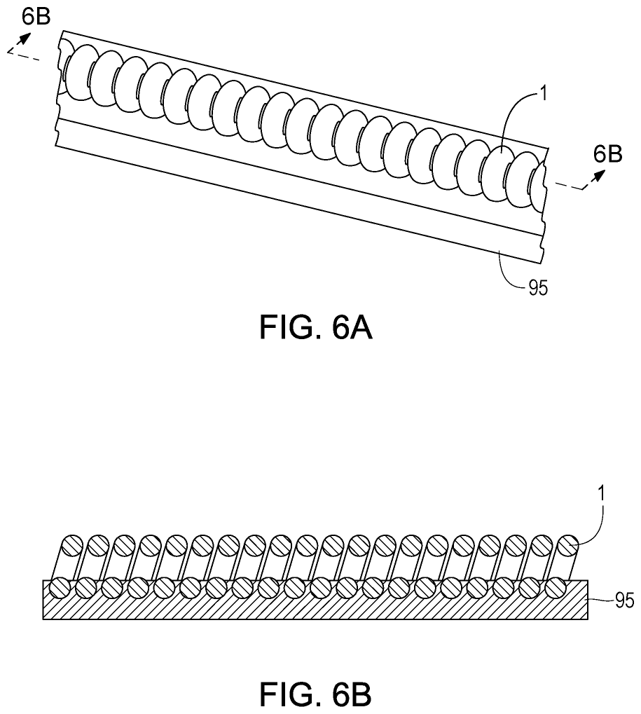

FIG. 6A is a perspective drawing of a high surface area electrode 92 (here a helical wire rope structure 1 which has the same detail as shown in the image in FIG. 1 herein) which is embedded in a polymer bottom layer, and FIG. 6B is a section view at line 6B-6B in FIG. 6A.

### DBS-Like

For an electrode assembly which is similar to prior art deep brain stimulation (DBS) electrodes, the high surface area electrode interface replaces the rings on traditional DBS electrodes which have a smooth surface. The same electrode ring design for DBS electrodes has been applied to spinal cord and peripheral neurostimulation. FIG. 7A is a perspective view of a portion of a DBS-like electrode with a high surface area electrode replacing the smooth surface rings, small disks or segmented electrodes in a prior art DBS electrode, as shown in FIGS. 5-7 of U.S. patent application no. 16/439,323. The prior art rings, disks, or segmented electrodes are replaced with high surface area electrodes to achieve a high surface area for a large charge transfer surface area between the conductive material and the target tissue. FIG. 7B is a section view at line 7B-7B in FIG. 7A. The high surface area electrode 1, in one embodiment, is secured to a rod or a tube 98, with partial embedding in a polymer substrate and/or recessed in a smaller diameter section 99 of the rod or tube.

### Ablation

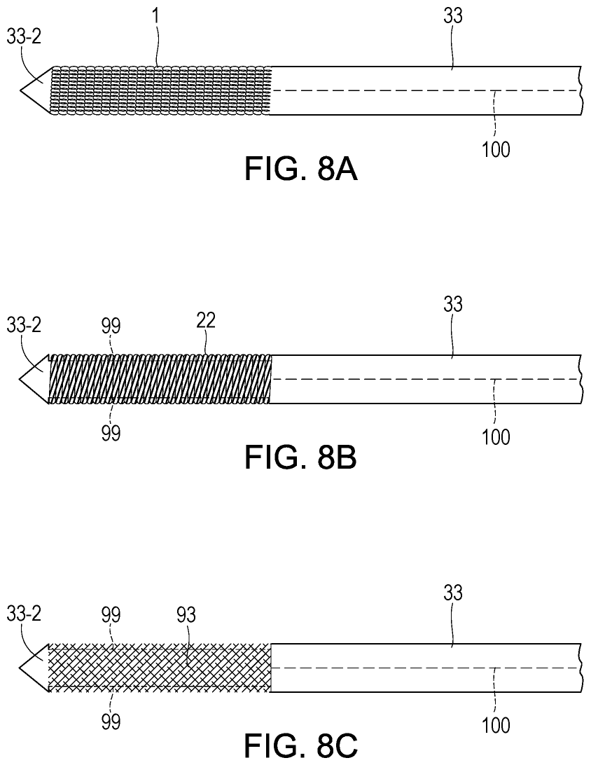

Various embodiments of the invention are shown in FIGS. 8A-8C with a high surface area electrode (in FIG. 8A, a helical wire rope structure) wound around the shaft of a needle 33 as in FIG. 8A. Instead of a helical wire rope structure, in FIG. 8B a wire rope 22 is wound around the shaft of the needle 33 to form the electrode interface, and in FIG. 8C a wire mesh 93 is wound around the shaft of the needle to form the electrode interface. The wires may be interspersed with non-conductive threads to generate additional porosity, density and charge injection capacity (surface area). To enable a constant diameter of the macroscopic dimensions of the needle as a whole, in one embodiment the needle tip 33-2 has an initial larger diameter which is recessed to a smaller diameter 99 to make space for the high surface area electrode as shown in FIGS. 8A-8C. That is, the high surface area electrode 1, 22, 93 is recessed in the section with a smaller diameter 99 so that the outer surface of the high surface area electrode, as secured, is essentially flush with the larger diameter of the needle shaft 33. Seating the electrode in this way makes it more secure and less likely to snag on tissue. In various embodiments, the electrode interface is secured to the needle by means of a process selected from the group consisting of welding, brazing gluing, compression fitting, and swaging. In another embodiment there is an outside coating formed from either electrically conductive or electrically non-conductive material. In another embodiment with a hollow needle there are ports allowing constant fluid immersion for the electrode interface. The electrode is connected to a power source by a wire 99 or other conductive pathway which, in one embodiment, is inside the needle shaft. A hollow needle shaft may also be used to provide fluids (such as sterile 0.9% saline or others) to ensure that the electrode interface is always in contact with liquid and no dry spots are present during the transfer process of electrical energy to and from electrode to tissue and back.

### PNS Electrodes, Continued

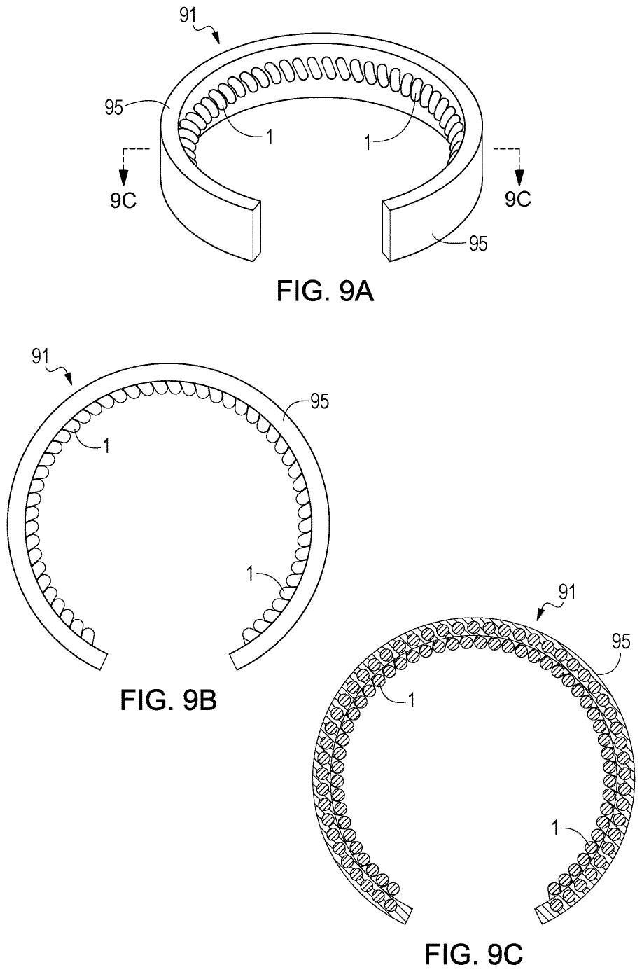

FIGS. 9A-9B are perspective views of a cuff-style high surface area electrode assembly 91 comprising the high surface area electrode 92 embedded in a nonconductive layer or length. FIG. 9C is a section view of the cuff electrode in FIG. 9A at line 9B-9B. In all figures the cuff is depicted as it would encircle a tissue target such as a peripheral nerve, cranial nerve or nerves on the outside of blood vessels such as blood vessels leading to the kidney or other organs of the body.

Embodiments of the device may have the wire structure functionalized with electrochemically active coatings to enable ion-selective sensing with very large surface area electrodes in a very small volume or space.

1. An implantable high surface area electrode assembly for comprising:
at least one element of nonconductive material selected from the group of a layer of nonconductive material and a length of nonconductive material; and
at least one high surface area electrode, said high surface area electrode being porous and being affixed securely to said element of nonconductive material.

2. The electrode assembly of claim 1 wherein said at least one element of nonconductive material includes at least one element fabricated from a polymer.

3. The electrode assembly of claim 1 wherein said high surface area electrode includes at least one electrode structure selected from the group consisting of a helical wire rope structure, a wire rope, a wire mesh, and a non-helical wire structure formed by at least one technique selected from the group of folding, rolling, twisting, and braiding.

4. The electrode assembly of claim 1 wherein said at least one element of nonconductive material further comprises:
a bottom layer of nonconductive material and a top layer of nonconductive material having an open window therein, and
wherein said high surface area electrode is configured relative to said open window so as to allow exposure of said high surface area electrode to at least one of bodily tissue and fluid via said open window in said top layer.

5. The electrode assembly of claim 4 wherein said high surface area electrode is partially embedded in at least one of the bottom layer and the top layer.

6. The electrode assembly of claim 1 wherein said at least one element of nonconductive material further comprises:
a length of nonconductive material, and
wherein said high surface area electrode is at least partially wound around said length of nonconductive material.

7. The electrode assembly of claim 6 wherein said length of nonconductive material about which said high surface area electrode is wound is formed as a smaller diameter section of an elongated structure of nonconductive material.

8. The electrode assembly of claim 2 wherein said polymer is selected from the group of silicone, polyether ether ketone (PEEK), parylene C, and polyimide.

9. The electrode assembly of claim 3 wherein at least some wires of said high surface area electrode form loops having a size between 0.00001 mm and 0.5 mm.

10. The electrode assembly of claim 1 wherein said at least one element of nonconductive material and said least one high surface area electrode are configured to provide an electrode from the group consisting of:
electrode portions of cylindrical needles,
electrode portions of leads,
electrode portions of paddle leads as used in spinal cord stimulation (SCS), peripheral nerve stimulation (PNS) and deep brain stimulation (DBS) applications,
electrode portions of stimulators as used in SCS, PNS or DBS, and cranial nerve applications such as vagal nerve stimulation (VNS),
electrode portions of electrocorticogram (ECoG) electrodes
electrode portions of leads for cardiac applications, and
electrode portions of stimulators for cardiac applications.

11. An implantable high surface area electrode assembly comprising:
at least one elongated element selected from the group of a rod and a tube; and
at least one high surface area electrode, said high surface area electrode being porous and being at least partially wound around and affixed securely to said elongated element.

12. The electrode assembly of claim 11 wherein said elongated element about which said high surface area electrode is wound is formed as a smaller diameter section of an elongated structure.

13. The electrode assembly of claim 12 wherein said elongated element diameter is selected relative to said high surface area electrode such that, when said high surface area electrode is wound thereabout, it has a resulting outer surface that is flush with the remainder of said elongated structure.

14. The electrode assembly of claim 11 wherein the said high surface area electrode includes at least one electrode structure selected from the group consisting of a helical wire rope structure, a wire rope, a wire mesh, and a non-helical wire structure formed by at least one technique selected from the group of folding, rolling, twisting, and braiding.

15. The electrode assembly of claim 14 wherein said elongated element is conductive and said high surface area electrode is attached to said elongated element by at least one technique selected from the group of welding, brazing gluing, compression fitting, and swaging.

16. The electrode assembly of claim 11 wherein said high surface area electrode is partially embedded in said elongated element.

17. electrode assembly of claim 11 wherein said high surface area electrode comprises wires interspersed with non-conductive threads.
